(19) United States
(12) Patent Application Publication
(10) Pub. No.: US 2021/0262967 A1
(43) Pub. Date: Aug. 26, 2021

(54) MICRO-HOTPLATE AND MEMS GAS SENSOR

(71) Applicants: OMRON Corporation, Kyoto-shi, Kyoto (JP); Figaro Engineering Inc., Minoo-shi, Osaka (JP)

(72) Inventors: Yoshitaka TATARA, Kyoto (JP); Masahito HONDA, Kyoto (JP); Hiroaki NAGAI, Kyoto (JP); Yoshiki ASHIHARA, Kyoto (JP); Kuniyuki IZAWA, Minoo-shi (JP); Masakazu SAI, Minoo-shi (JP); Kenichi YOSHIOKA, Minoo-shi (JP)

(21) Appl. No.: 16/972,754

(22) PCT Filed: Feb. 22, 2019
(86) PCT No.: PCT/JP2019/006748

§ 371 Date: Dec. 7, 2020

(30) Foreign Application Priority Data
Jun. 8, 2018 (JP) 2018-110043

(51) Int. Cl. G01N 27/12; G01N 33/00; H05B 3/20

(52) U.S. Cl. CPC G01N 27/128; G01N 27/125; H05B 2203/016; H05B 3/20; H05B 2203/003; G01N 33/0047

(57) ABSTRACT

A micro-hotplate comprises a Si substrate having a cavity and a support layer over the cavity; at least an electrode; and a heater, both provided on the support layer. The electrode surrounds the heater and the heater is disposed inside the electrode. The power efficiency of the micro-hotplate is improved.

### FIELD OF THE INVENTION

The present invention relates to a micro-hotplate fabricated by MEMS technology and a gas sensor including the micro-hotplate and, in particular, to the disposition of the heater and the electrodes in the micro-hotplate.

### BACKGROUND ART

Micro-hotplates have been used for gas sensors and so on; for example, micro-hotplates provided with a gas sensitive layer become gas sensors. It has been considered to be preferable that the heater in a micro-hotplate surrounds a gas sensitive layer and its electrodes, namely, that the heater is disposed outside the electrodes. This construction has been considered useful for reducing the temperature distribution within the gas sensitive layer (Patent Document 1: WO2005/012892). According to WO2005/012892, within a concentric three-track heater, a pair of comb-teeth-like electrodes and the gas sensitive layer is provided.

There are examples not obeying this principle that the heaters are disposed at the outside of the gas sensing layers. For example, Patent Document 2 (US2018/0017516) discloses a gas sensor where four sets of the combination of gas sensitive layers and their electrodes are provided at the outside of a heater.

### Prior Document List

### Patent Document

Patent Document 1: WO2005/012892

Patent Document 2: US2018/0017516

### SUMMARY OF THE INVENTION

### Problem to be Solved by the Invention

The inventors have found that when the heater and the electrodes are disposed such that the electrodes surround the heater, or the heater is disposed inside the electrodes, then, the power efficiency of the micro-hotplates is improved. For example, this configuration gives higher gas sensitivity with the same power consumption.

The object of the invention is to improve the power efficiency of micro-hotplates and MEMS gas sensors using the same.

A micro-hotplate according to the invention comprises a Si substrate having a cavity and a support layer over the cavity; at least an electrode; and a heater, both on the support layer. The electrode surrounds the heater, and the heater is disposed inside the electrode.

A MEMS gas sensor according to the invention comprises: a Si substrate having a cavity and a support layer over the cavity; at least an electrode; a heater; and a gas sensitive layer, and the electrode, the heater, and the gas sensitive layer are provided on the support layer. The electrode surrounds the heater, the heater is disposed inside the electrode, and the gas sensitive layer covers the electrode.

The micro-hotplates according to the invention are also usable for applications other than gas sensors. In the present specification, descriptions about micro-hotplates apply to MEMS gas sensors as they are. In the following, MEMS gas sensors will be simply referred to as gas sensors.

FIGS. 8 and 9 indicate the gas sensitivity of gas sensors where the heater is disposed inside and the electrodes are disposed outside. FIG. 10 indicates the gas sensitivity of a conventional gas sensor where the heater is disposed outside and the electrodes are disposed inside. These gas sensors were driven with the same power consumption. As a general empirical rule, the sensitivity of gas sensors to fuel gases such as iso-butane improves when the temperature of the gas sensitive layers is increased. The gas sensors in FIGS. 8 and 9 (embodiments) had substantial iso-butane sensitivity, on the contrary, while the gas sensor in FIG. 10 (conventional example) had little sensitivity to iso-butane. Further, while the gas sensor in FIG. 10 had remarkably lower sensitivity to ethanol than that to hydrogen, the gas sensors in FIGS. 8 and 9 had substantially equal sensitivity to both ethanol and hydrogen. These facts show that the power efficiency of micro-hotplates is improved by disposing the heater inside and the electrodes outside such that the electrodes surround the heater.

Preferably, the electrode has a ring-like shape having an opening, the heater has a disclike or annular heat generating region, and both ends of the heater are connected to a pair of heater leads which are drawn out through the opening. Since the heater is disclike or annular, the generated heat flows uniformly towards the electrode. Further, the heater leads can be drawn out through the opening. In the specification, a disc and a disclike shape mean both a circle and its interior.

More preferably, the heat generating region is disclike. The heater folds back at plural times within the heat generating region and is provided with an arc-like portion along an outer periphery of the heat generating region. More preferably, the heater is linear between the fold-back positions. When arranging the heater within the heat generating region with the plural fold-backs, it becomes difficult to draw out both ends of the heater through one opening. For dealing with this problem, the arc-like portion is provided along the outer periphery of the heat generating region, and consequently both ends of the heater can be drawn out through the same opening.

Particularly preferably, the electrode comprises at least two electrodes opposing each other. The at least two electrodes have ring-like shapes surrounding the heater and have a common opening. As an alternative, it is possible to provide only one electrode and to use the heater as another electrode. However, in this configuration, the electrical potential within the heater becomes not constant and this makes the processing of the signal from the gas sensitive layer difficult. Therefore, the two electrodes facing each other are made a ring-like shape surrounding the heater, and the heater leads are drawn out through the common opening.

Preferably, the electrodes consist of two electrodes, and the two electrodes are connected to two electrode leads. These outer periphery of the heat generating region is divided, along an open circle from the common opening to the common opening, into three portions by two connection points between the two electrode leads and the two electrodes. In this case, the micro-hotplate further comprises an arc-like dummy electrode on the support layer, not connected to the two electrodes, and disposed along the outer periphery of the heat generating region, from the common opening to one of the two connection points. The dummy electrode and the proper two electrodes surround almost uniformly the outer periphery of the heater from the common opening to the common opening. In addition, when applying a paste to form the gas sensitive layer on the support layer, the dummy electrode restricts the expansion of the paste.

When the electrodes are provided on an insulating layer covering the heater, the heater and the electrodes can be disposed without any restriction. However, when the electrodes and the heater are disposed at the same height with reference to a support layer, namely, when the electrodes and the heater are formed at the same time, the disposition of them is limited to whether the heater is inside (conventional) or the heater is inside (the invention). Thus, the invention is particularly suitable when the electrodes and the heater are provide at the same height with reference to a support layer.

### BRIEF DESCRIPTION OF THE ACCOMPANYING DRAWINGS

FIG. 1 A plan view of a micro-hotplate according to an embodiment.

FIG. 2 A plan view of a micro-hotplate according to a second embodiment.

FIG. 3 A plan view of a micro-hotplate according to a modified embodiment.

FIG. 4 A plan view of a micro-hotplate according to a third embodiment.

FIG. 5 A sectional view of a MEMS gas sensor having the micro-hotplate in FIG. 1.

FIG. 6 A sectional view of a MEMS gas sensor having the micro-hotplate in FIG. 4.

FIG. 7 A plan view of a conventional micro-hotplate.

FIG. 8 A characteristic view indicating the gas sensitivity of the MEMS gas sensor having the micro-hotplate in FIG. 1.

FIG. 9 A characteristic view indicating the gas sensitivity of the MEMS gas sensor having the micro-hotplate in FIG. 2.

FIG. 10 A characteristic view indicating the gas sensitivity of the MEMS gas sensor having the conventional micro-hotplate.

The best embodiment for carrying out the invention will be described.

### EMBODIMENTS

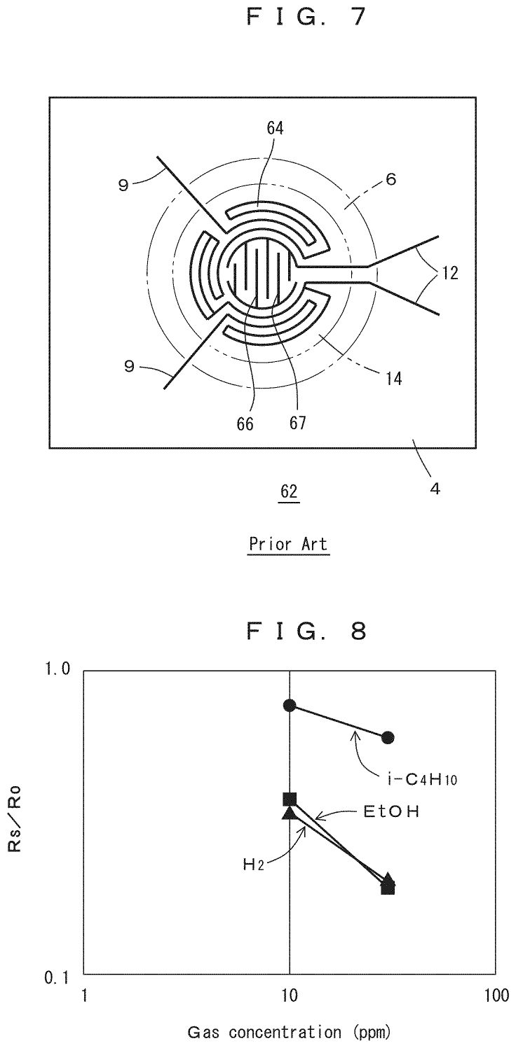

FIGS. 1 to 6 indicate micro-hotplates 2, 22, 32, 42 according to the embodiments and gas sensors 40, 45 provided with them. Micro-hotplates will be simply referred to as “hotplates” in the following. Hot-plates according to a conventional example 62 is indicated in FIG. 7. The same symbols in the embodiments in FIGS. 1 to 6 refer to the same thing, and the descriptions regarding the embodiment in FIG. 1 apply to other embodiments unless otherwise specified.

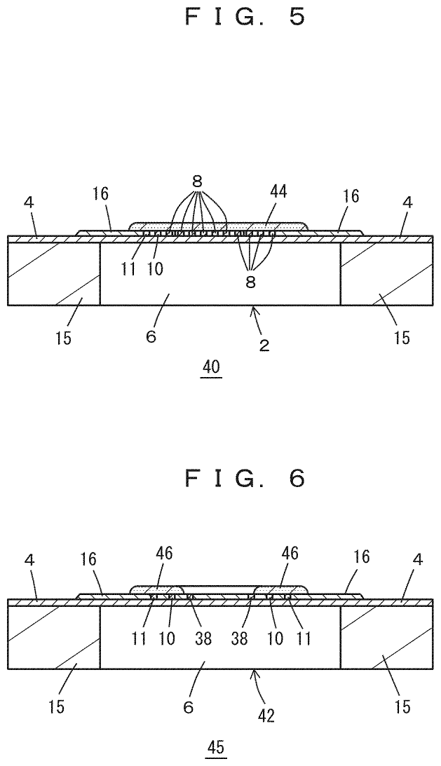

In FIG. 1, the hotplate 2 is provided on a support layer 4 on a Si substrate 15 in FIG. 5. The support layer 4 is electrically insulating and comprises, for example, three layers of Si oxide, Si nitride, and Si oxide, but the materials and structure of the support layer 4 are arbitrary. The support layer 4 is in a diaphragm-like shape covering a cavity 6 but it may be a bridge over the cavity 6. The materials for the heater 8 may be a metal such as Pt or an electrically conductive Si with dopants. The heater 8 folds back at plural times within a disclike heat generating region comprising a circle and its interior. The heater is linear between fold-back positions, and, in addition, is provided with a semi-circular arc-like portion 19 returning form a left portion to a right portion in FIG. 1. Further, both ends of the heater 8 are drawn out as a pair of heater leads 9, 9 through an opening 17 between electrodes 10, 11 to the outside of the cavity 6 and are connected to pads not shown.

Since the heat generating region including the arc-like portion 19 is disclike, the generated heat flows uniformly from the heater 8 to the side of the electrodes 10, 11. As shown in FIG. 1, the heater 8 is disposed within the heat generating region with plural fold-backs from the right to the left, and, for connecting the left end of the heater 8 to the heater lead 9, the arc-like portion 19 is provided. Since the heater leads 9, 9 can not cross the electrodes 10, 11, they are drawn out through the common opening 17.

The pair of the electrodes 10, 11 surround the heater 8 as a dual circle having the opening 17 and they face each other. Since the electrodes 10, 11 can not cross each other, there is a region where the outer electrode 11 can not be provided. Preferably, a dummy electrode 13 is provided within the region to make the heat flow from the electrodes 10, 11 to their outside more uniformly, regardless of the positions around the circle. However, the dummy electrode 13 may not be provided. The electrodes 10, 11 are connected to electrode leads 12, drawn out through the cavity 6, and connected to pads or the like not shown. On an area 14 for the gas sensitive layer, a gas sensitive layer is provided and it comprises a metal oxide semiconductor such as SnO2, In2O3, or WO3 is provided. The gas sensitive layer covers the electrodes 10, 11 and may be a thin layer or a thick layer.

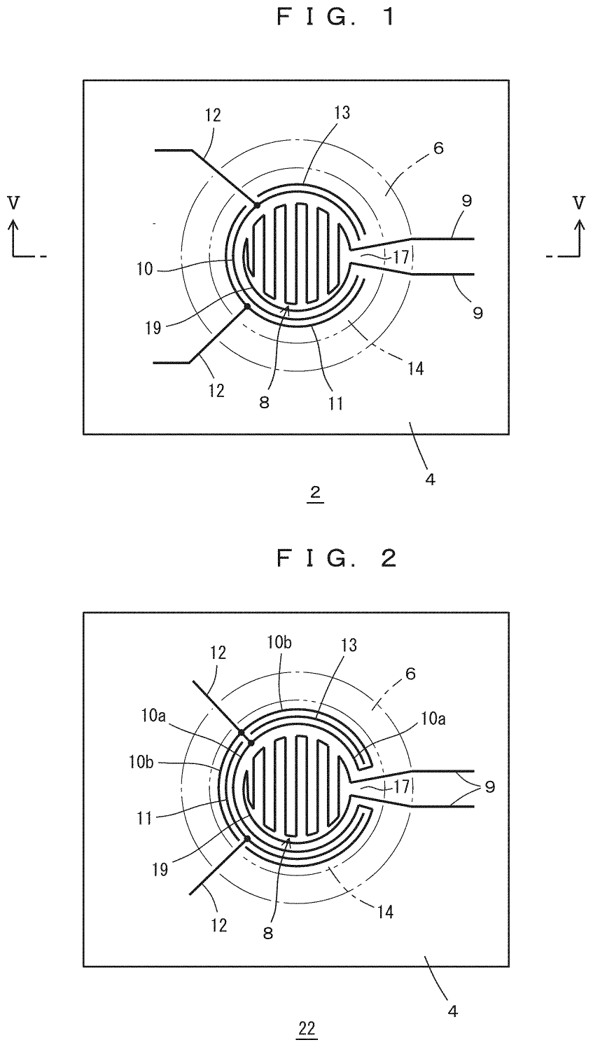

FIG. 2 indicates a hotplate 22 according to a second embodiment, and the electrode 10 comprises an inner electrode 10a and an outer electrode 10b and is a dual circular electrode. In addition, between the electrodes 10a, 10b, the electrode 11 is disposed. Regarding other points, it is the same to the embodiment in FIG. 1.

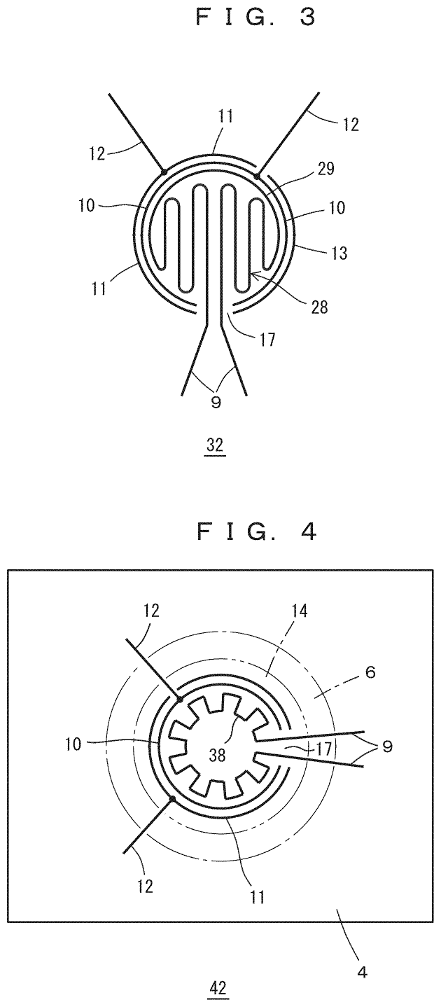

A hotplate 32 (modification) in FIG. 3 has the heater 28 and the arc-like portion 29 within the heat generating region that have a different arrangement from those in FIGS. 1, 2.

FIG. 4 indicates a hotplate 42 according to a third embodiment, this hotplate has an annular heater 38, and the heater has not the arc-like portion. Regarding other points, it is the same to the embodiment in FIG. 1.

FIG. 5 indicates a gas sensor 40 including the hotplate 2 in FIG. 1; there is provided a gas sensitive layer 44 and it comprises a thin film or a thick film of metal oxide semiconductor, such as SnO2, In2O3, or WO3. In addition, the support layer 4 provided on a Si substrate 15 is indicated. Further, the heater 8 and the electrodes 10, 11 are formed at the same time with the same mask and are at the same height with reference to the support layer 14. Around the heater 8 and the electrodes 10, 11, an insulating layer 16 is provided, however, the insulating layer 16 may not be provided.

FIG. 6 indicates a gas sensor 42 including the hotplate 42 in FIG. 4; it has a ring-like gas sensitive layer 46 covering the electrodes 10, 11, and a portion of the heater 38 is exposed. Regarding other points, it is the same to the embodiment in FIG. 1.

FIG. 7 indicates a conventional micro-hotplate 62; at the inside of a ring-like heater 64, a pair of comb-teeth-like electrodes 66, 67 are provided. Regarding other points, it is the same to the embodiment in FIG. 1.

### Gas Sensitivity

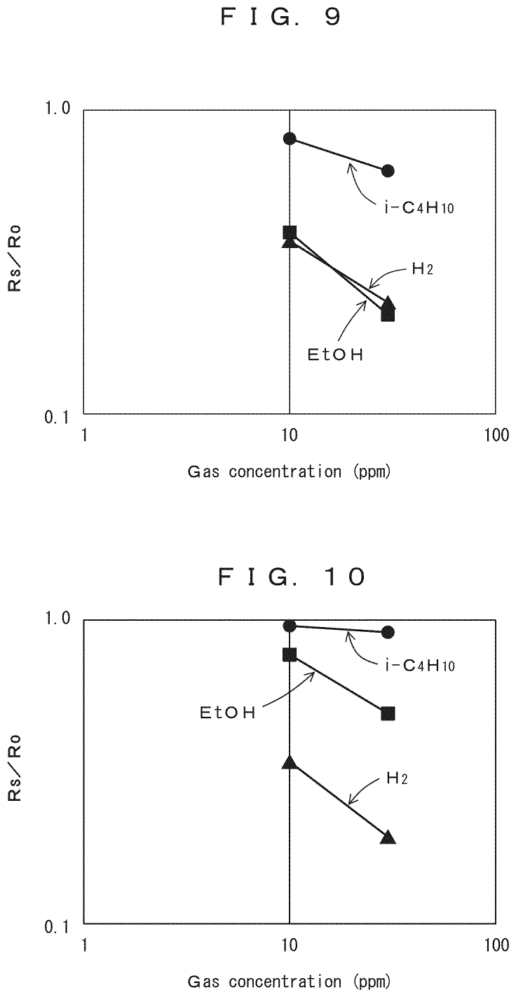

SnO2 paste was dispensed on the area 14 for the gas sensitive layer of a micro-hotplate. Then the paste was baked at 600 degree Celsius in air to prepare the gas sensitive layer 44 comprising a SnO2 thick film (film thickness of about 20 micro-meter) to fabricate gas sensors. These gas sensors were continuously heated to 350 degree Celsius at the gas sensitive layer, and the resistance was measured in iso-butane, ethanol, and hydrogen at 10 ppm and 30 ppm. The same power was applied to the gas sensors. FIG. 8 indicates the results of the micro-hotplate 2 in FIG. 1, FIG. 9 indicates the results of the micro-hotplate 22 in FIG. 2, and FIG. 10 indicates the results of the conventional micro-hotplate 62 in FIG. 7. The results are shown as an average of five gas sensors used for the measurement. Rs/R0 in the figures indicate the ratios of resistance between gas and air.

In FIG. 10 (conventional example), the iso-butane sensitivity was little and the ethanol sensitivity was low. In contrast to them, in the embodiments in FIGS. 8 and 9, substantial iso-butane sensitivity was generated and the ethanol sensitivity was equivalent to the hydrogen sensitivity. These facts indicate that the gas sensitive layers 44 in the embodiments were heated more efficiently at the same power than that of the conventional example. They have high ethanol sensitivity suitable for alcohol detection and have iso-butane sensitivity so that they can be applied for hydrocarbon detection.

### LIST OF SYMBOLS

2, 22, 32, 42 micro-hotplate
4 support layer
6 cavity
8,28 heater
9 heater lead
10, 11 electrode
10a, 10b electrode
12 electrode lead
13 dummy electrode
14 area for the gas sensitive layer
15 Si substrate
16 insulating layer
17 opening
19, 29 arc-like portion
38 heater
40, 45 gas sensor
44, 46 gas sensitive layer
62 micro-hotplate
64 heater
66, 67 electrode

1-8. (canceled)

9. A MEMS gas sensor comprising: a Si substrate having a cavity and a support layer over the cavity; two electrodes facing each other; one heater; two heater leads connected to both ends of said heater; and a gas sensitive layer, all on the support layer,
wherein said two electrodes have a dual ring-like shape having a common opening and surround said heater,
wherein said heater is disposed and folds back at plural times inside said two electrodes and forms a disclike or annular heat generating region,
wherein said two heater leads are drawn out through said common opening, and
wherein said gas sensitive layer covers said two electrodes.

10. The MEMS gas sensor according to claim 9, wherein said two electrodes and said heater are provided at the same height with reference to said support layer.

11. The MEMS gas sensor according to claim 9, wherein said heat generating region is disclike, and
wherein said heater is provided with an arc-like portion along outer periphery of said heat generating region.

12. The MEMS gas sensor according to claim 11, further comprising two electrode leads connected to said two electrodes; and an arc-like dummy electrode, all on the support layer,
wherein the outer periphery of the heat generating region is divided, along an open circle from said common opening to the common opening, into three portions by two connection points between the two electrode leads and the two electrodes, and
wherein said arc-like dummy electrode is not connected to the two electrodes and is disposed along the outer periphery of the heat generating region from said common opening to one of the two connection points.
